SEQUENCE LISTING (5 sequences)

SEQ ID NO: 1 - DNA, 4818 bp, synthetic construct
  misc_feature 1..4818: note = pGL3-Basic sequence of the coding sequence for the luciferase expression plasmid
  source 1..4818: mol_type = other DNA
ggtaccgagctcttacgcgtgctagcccgggctcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 2 - DNA, 200 bp, synthetic construct
  misc_feature 1..200: note = BAZ1A promoter region sequence
  source 1..200: mol_type = other DNA
ctagatcttttggctgataatctcaaacatggaggatgcttctgaatcttcacgaggggttgctccattaattaataatgtagttctcccaggctctccgctgtctcttcctgtatcagtgacaggctgtaaaagtcatcgagtagccaataaaaaggtagaagcgaggagtgaaaagctcctcccaacagctcttcctc

SEQ ID NO: 3 - DNA, 200 bp, synthetic construct
  misc_feature 1..200: note = ZHX2 promoter region sequence
  source 1..200: mol_type = other DNA
atttactgcccccctaccttggccagtaaccagcgcctttagggctagcctcccccccagctcctgcctgaaaaatgacatttcgccggtgtctccggagggggctgaatttcactttgtaactttctgcggaacccgagcccgggtggcagctcgggtggtggtatcgtatgcaaatacgcatgctgacgttacagatc

SEQ ID NO: 4 - DNA, 200 bp, synthetic construct
  misc_feature 1..200: note = MYC promoter region sequence
  source 1..200: mol_type = other DNA
caccggccctttataatgcgagggtctggacggctgaggacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatct

SEQ ID NO: 5 - DNA, 200 bp, synthetic construct
  misc_feature 1..200: note = NFKBIE promoter region sequence
  source 1..200: mol_type = other DNA
aggagtgagtcaaggcccgggaaccacagactccaagcctacgcagagcccgggaagggggattccggaggggcggggcctctttccggaagcgcccgccgggggcggggagggggcggggccatccgcgtgaggcgaccctgttggtccggaggggcggggcgaggaggaggacccgcttgggcggttcggctgcccac

(19) United States
(12) Patent Application Publication
(10) Pub. No.: US 2026/0115245 A1
(43) Pub. Date: Apr. 30, 2026

(54) USE OF MUCOR RACEMOSUS AND METABOLITE THEREOF IN PREPARATION OF DRUG FOR PREVENTING AND/OR TREATING ULCERATIVE COLITIS

(71) Applicant: Nankai University, Tianjin (CN)

(72) Inventors: Xingzhong LIU, Tianjin (CN); Jia LIU, Tianjin (CN); Huiwen XIAO, Tianjin (CN); Yue CHEN, Tianjin (CN); Jiamin ZHAO, Tianjin (CN)

(21) Appl. No.: 19/334,849

(22) Filed: Sep. 20, 2025

(30) Foreign Application Priority Data
Oct. 31, 2024 (CN) 202411533781.0

(51) Int. Cl. A61K 36/06; A61K 31/513; A61P 1/04

(52) U.S. Cl. CPC A61K 36/06; A61K 31/513; A61P 1/04

(57) ABSTRACT

The present invention relates to the use of Mucor racemosus and a metabolite thereof in the preparation of a drug for preventing and/or treating ulcerative colitis, which belongs to the technical field of biopharmaceuticals. Mucor racemosus CGMCC NO: 40320 and the metabolite 5mc (5-methylcytosine) thereof of the present disclosure can significantly mitigate problems of disease index, body weight loss and reduction in index of immune-related organs (e.g., spleen) in mice with ulcerative colitis disease, can alleviate the occurrence of inflammation in colon tissue, thereby improving the intestinal wall integrity of intestinal tissues, and thus can be used in the preparation of a drug for preventing and/or treating ulcerative colitis. The present disclosure expands the application fields of Mucor racemosus and provides guidance for clinical application of Mucor racemosus in the treatment of colitis.

### CROSS-REFERENCE TO RELATED APPLICATION

This patent application claims priority to and benefit of Chinese Patent Application No. 2024115337810 filed with the China National Intellectual Property Administration on Oct. 31, 2024, the disclosure of which is incorporated by reference herein in its entirety as part of the present application.

### REFERENCE TO SEQUENCE LISTING

A computer readable XML file entitled “GWP20250701575_sequence_listing”, which was created on Sep. 8, 2025 and has a file size of about 12,939 bytes, contains the sequence listing for this application and is hereby incorporated by reference in its entirety.

### TECHNICAL FIELD

The present disclosure belongs to the technical field of biopharmaceuticals, and in particular relates to the use of Mucor racemosus and a metabolite thereof in the preparation of a drug for preventing and/or treating ulcerative colitis.

### BACKGROUND

Ulcerative colitis (UC) is a non-specific chronic refractory intestinal inflammation, which is at risk of developing cancer in the case of being refractory to long-term treatment, with the canceration rate increasing gradually with the duration of the disease. The clinical manifestations of ulcerative colitis mainly include diarrhea, mucopurulent bloody stools and abdominal pain. The disease is characterized by alternating recurrence and remission, which seriously affects the quality of life of patients. In severe cases or when complications develop, the disease may become life-threatening. At present, the treatment of ulcerative colitis mainly relies on drug intervention. Drugs for treating ulcerative colitis mainly include anti-inflammatory agents, immunomodulators, biological agents etc., such as 5-aminosalicylic acid, glucocorticoids and monoclonal antibodies. However, these drugs have certain limitations, such as unstable efficacy and high recurrence rate after discontinuation, side effects such as liver and kidney function damage and even bone marrow suppression, and shortcomings such as increased risk of potential infections and high treatment costs. Therefore, clinically, there is a lack of safe, stable, effective and universally applicable drugs for treating ulcerative colitis.

The exact cause of ulcerative colitis remains unclear and may be related to multiple factors such as genetics, immunity, environment and infection. However, numerous studies have shown that it is related to the pathological interaction between the immune system and intestinal commensal bacteria. Intestinal microbial dysbiosis often leads to a high incidence of ulcerative colitis. Intestinal microorganisms play a beneficial role in regulating immune responses and ameliorating ulcerative colitis and other infectious diseases. Moreover, this beneficial effect is not limited to intestinal tissues but can also extend to extraintestinal organs, enhancing the responsiveness of immune cells and thus helping to treat extraintestinal diseases. For example, Bifidobacterium adolescentis can alleviate colitis by inducing protective Treg and Th2 cells (Fan, Lina et al. “B. adolescentis ameliorates chronic colitis by regulating Treg/Th2 response and gut microbiota remodeling.” Gut microbes vol. 13, 1 (2021): 1-17. doi: 10.1080/19490976.2020.1826746). Chinese patent publication CN 118497080A discloses that ulcerative colitis in mice is alleviated by regulating the level of intestinal oxidative stress and improving the structure of the intestinal microbiota by using fermented Lactobacillus mucosae. In addition to the use of microorganisms themselves, there have also been studies that use metabolites of microorganisms to regulate immune responses. For example, tryptophan indole derivatives produced by Lactobacillus reuteri can stimulate intestinal differentiation to produce CD4⁺CD8αα⁺ double-positive intraepithelial lymphocytes, thereby ameliorating inflammatory bowel disease (Cervantes-Barragan, Luisa et al. “Lactobacillus reuteri induces gut intraepithelial CD4⁺CD8αα⁺ T cells.” Science (New York, N.Y.) vol. 357, 6353 (2017): 806-810. doi: 10.1126/science.aah5825), and so on. Although the above microorganisms and microbial metabolites exhibit immense potential in alleviating ulcerative colitis, the safety of their clinical application has not yet been effectively confirmed.

In addition to the probiotics mentioned above, the microorganisms in the intestine also include fungi. Although fungi account for only 0.01-0.1% of the intestinal microbiota, they play an indispensable role in maintaining the dynamic balance of the intestinal microbiota. However, there are currently no studies on fungi and metabolites thereof in the treatment of enteritis.

### SUMMARY

In view of this, an objective of the present disclosure is to provide the use of Mucor racemosus and a metabolite thereof in the preparation of a drug for preventing and/or treating ulcerative colitis, so as to expand the application fields of Mucor racemosus and provide a safe, reasonable and effective drug for the clinical treatment of ulcerative colitis.

To achieve the above objective of the present disclosure, the present disclosure provides the following technical solutions:

The present disclosure provides the use of Mucor racemosus or a formulation containing Mucor racemosus in the preparation of a drug for preventing and/or treating ulcerative colitis, where the deposit number of Mucor racemosus is CGMCC NO: 40320.

The present disclosure further provides the use of a metabolite of Mucor racemosus or a formulation containing the metabolite of Mucor racemosus in the preparation of a drug for preventing and/or treating ulcerative colitis, where the metabolite of Mucor racemosus includes 5-methylcytosine.

Further, the drug is any one or more of the following drugs:
1) the drug is a drug for alleviating body weight loss in an ulcerative colitis patient;
2) the drug is a drug for increasing colon length of an ulcerative colitis patient;
3) the drug is a drug having an immunomodulatory effect;
4) the drug is a drug having an anti-inflammatory effect;
5) the drug is a drug having an antioxidant capacity; and
6) the drug is a drug for enhancing intestinal wall integrity of colon tissue.

The drug having an immunomodulatory effect in 3) can reduce spleen index in an ulcerative colitis patient.

The drug having an anti-inflammatory effect in 4) can reduce the content of any one or several of IL-6, IL-1β and TNF-α in ulcerative colitis tissue.

The drug having an antioxidant capacity in 5) can reduce the content of reactive oxygen species (ROS) in ulcerative colitis tissue.

The drug for enhancing intestinal wall integrity of colon tissue in 6) can increase the expression quantity of Claudin-2 in ulcerative colitis tissue.

The present disclosure further provides a drug for preventing and/or treating ulcerative colitis, where the drug includes one or two of Mucor racemosus and 5-methylcytosine.

In the present disclosure, the ulcerative colitis includes ulcerative colitis in remission phase, mild ulcerative colitis, moderate ulcerative colitis, severe ulcerative colitis or acute severe ulcerative colitis.

Preferably, the drug for preventing and/or treating ulcerative colitis includes an oral formulation.

The present disclosure provides the use of Mucor racemosus and a metabolite thereof in the preparation of a drug for preventing and/or treating ulcerative colitis, where a deposit number of Mucor racemosus is CGMCC NO: 40320. The present disclosure has demonstrated, through experiments on an animal model of ulcerative colitis, that Mucor racemosus and the metabolite 5mc (5-methylcytosine) thereof have a good effect on ulcerative colitis. Experiments have confirmed that Mucor racemosus and 5mc can mitigate the problem of body weight loss in ulcerative colitis mice and increase the colon length of ulcerative colitis mice. Meanwhile, Mucor racemosus and 5mc have an immunomodulatory capacity, can reduce the spleen index and disease index of colitis mice, and thus reducing alleviating the symptoms and severity of colitis. In addition, Mucor racemosus and the metabolite 5mc thereof have a certain anti-inflammatory effect, and can significantly reduce the contents of ROS, IL-6, IL-1β and TNF-α in ulcerative colitis colon tissue. Moreover, Mucor racemosus and 5mc can significantly increase the expression quantity of Claudin-2 in colitis tissue, thereby enhancing the intestinal wall integrity of colon tissue. The present disclosure reveals the pathways of action of Mucor racemosus and the metabolite in the prevention and/or treatment of ulcerative colitis, which provides important clues for exploring the impact of fungi and metabolites thereof on the body health, and also provides new ideas for their clinical application in the treatment of colitis.

The present disclosure further provides a drug for preventing and/or treating ulcerative colitis, which expands the application fields of Mucor racemosus and provides a novel, safe and effective drug for the treatment of ulcerative colitis.

### BRIEF DESCRIPTION OF THE DRAWINGS

FIG. 1: Impact of Mucor racemosus on body weight of colitis mice, in which *** indicates p<0.001;

FIG. 2: Impact of Mucor racemosus on colon length of colitis mice, in which indicates p<0.001;

FIG. 3: Impact of Mucor racemosus on the spleen of colitis mice, in which *** indicates p<0.001;

FIG. 4: Impact of Mucor racemosus on disease index of colitis mice, in which the disease index of the blank group is 0; the model group vs. the blank group, *** indicates p<0.001, and ns indicates no significant difference; the Mucor racemosus administration group vs. the blank group, # indicates p<0.05, ## indicates p<0.01, and ns indicates no significant difference;

FIG. 5: Impact of Mucor racemosus on ROS content in the colon of colitis mice, in which *** indicates p<0.001;

FIG. 6: Impact of Mucor racemosus on contents of IL-6, IL-1B and TNF-α in the colon of colitis mice, in which *** indicates p<0.001;

FIG. 7: Impact of Mucor racemosus on expression quantity of Claudin-2, a marker of intestinal wall integrity of the colon in colitis mice, in which *** indicates p<0.001;

FIG. 8: Statistical results of the content of metabolite 5mc in the culture medium for in-vitro culture of Mucor racemosus, in which *** indicates p<0.001;

FIG. 9: Statistical results of the content of 5mc in feces after intragastric administration of Mucor racemosus to colitis mice, in which *** indicates p<0.001;

FIG. 10: Impact of the metabolite 5mc of Mucor racemosus on body weight of colitis mice, in which *** indicates p<0.001;

FIG. 11: Impact of the metabolite 5mc of Mucor racemosus on colon length of colitis mice, in which *** indicates p<0.001;

FIG. 12: Impact of the metabolite 5mc of Mucor racemosus on disease index of colitis mice, in which the model group vs. the blank group, *** indicates p<0.001, and ns indicates no significant difference; the 5mc intragrastric administration group vs. the blank group, # indicates p<0.05, ## indicates p<0.01, and ns indicates no significant difference;

FIG. 13: Impact of the metabolite 5mc of Mucor racemosus on ROS content in the colon of colitis mice, in which *** indicates p<0.001;

FIG. 14: Impact of the metabolite 5mc of Mucor racemosus on contents of IL-6, IL1-β and TNF-α in the colon of colitis mice, in which *** indicates p<0.001;

FIG. 15: Impact of the metabolite 5mc of Mucor racemosus on expression quantity of Claudin-2, a marker of intestinal wall integrity of the colon in colitis mice, in which ** indicates p<0.001;

FIG. 16: Content of 5mc in the nucleus and cytoplasm after treatment of colon cell lines Caco-2 and HT-29 with the metabolite 5mc of Mucor racemosus for 48 hours, in which * indicates p<0.05, and *** indicates p<0.001;

FIG. 17: Construction of luciferase expression plasmids with promoter regions of different GC percentages (upper panel); and contents of luciferase in Caco-2 cells transfected with the above plasmids and treated with 5mc for 48 hours (lower panel), in which * indicates p<0.05, *** indicates p<0.001, and ns indicates no significant difference.

### DETAILED DESCRIPTION OF THE EMBODIMENTS

The present disclosure provides the use of Mucor racemosus or a formulation containing Mucor racemosus in the preparation of a drug for preventing and/or treating ulcerative colitis. In the present disclosure, the Mucor racemosus used is taxonomically named Mucor racemosus, which has been deposited in the China General Microbiological Culture Collection Center (CGMCC, address: NO. 1 West Beichen Road, Chaoyang District, Beijing 100101, China), with the following details: address of the depository institution: Institute of Microbiology, Chinese Academy of Sciences, No. 3, Yard 1, Beichen West Road, Chaoyang District, Beijing; deposit date: Sep. 19, 2022; and deposit number: CGMCC NO. 40320 (disclosed in patent CN 116286390A).

In the present disclosure, the formulation containing Mucor racemosus is a formulation containing a Mucor racemosus strain with CGMCC NO. 40320, which may be an oral formulation, such as a fungal suspension, a lyophilized powder, a tablet, a capsule, a pill or an oral solution, containing Mucor racemosus with CGMCC NO. 40320. In the present disclosure, the formulation containing Mucor racemosus further comprises a pharmaceutically acceptable excipient, including one or several of a diluent, a colorant, a sweetener, a coating agent, a binder, an absorbent, a disintegrant, a dispersant, a wetting agent, a co-solvent, a buffer and a surfactant.

The present disclosure further provides the use of a metabolite of Mucor racemosus or a formulation containing the metabolite of Mucor racemosus in the preparation of a drug for preventing and/or treating ulcerative colitis. The metabolite of Mucor racemosus is preferably 5-methylcytosine, a metabolite produced by Mucor racemosus with the deposit number of CGMCC NO. 40320. The formulation containing the metabolite of Mucor racemosus contains 5-methylcytosine. 5-methylcytosine is metabolically produced via the metabolism of Mucor racemosus with the deposit number of CGMCC NO. 40320, or may be prepared by chemical methods.

In the present disclosure, the C57BL/6J mouse model is used as the experimental material to verify the preventive and therapeutic effects of Mucor racemosus with CGMCC NO. 40320 and the metabolite 5-methylcytosine thereof on ulcerative colitis. In the present disclosure, there is no special restriction on the method for constructing the C57BL/6J mouse model. It is sufficient to construct a model of ulcerative colitis with dextran sulfate sodium well known in the art. The body weight, colon length, spleen index, and disease index of the mouse model of ulcerative colitis were calculated, and the content of reactive oxygen species (ROS) and inflammatory factors IL-6, IL-1β and TNF-α in colon tissue and the expression of Claudin-2, a marker of intestinal wall integrity of the colon, were detected. The results show that after 5 days of intragastric administration of Mucor racemosus or 5-methylcytosine, the ulcerative colitis mice exhibit a significant increase in body weight and colon length. With the extension of modeling time, the disease development index decreases significantly, the ROS content and contents of inflammation-related factors IL-6, IL1-β and TNF-α in colon tissue decrease significantly, and the expression quantity of Claudin-2, a marker gene of intestinal wall integrity of colon, increases significantly. Therefore, the Mucor racemosus or the formulation containing Mucor racemosus, and the metabolite 5-methylcytosine of Mucor racemosus or the formulation containing 5-methylcytosine of the present disclosure can effectively treat ulcerative colitis disease, have the effects of alleviating body weight loss in an ulcerative colitis patient and increasing colon length in an ulcerative colitis patient, have immunomodulatory, anti-inflammatory, and antioxidant capacities, and can also enhance the intestinal wall integrity of colon tissue.

The present disclosure further provides a drug for preventing and/or treating ulcerative colitis, where the drug includes one or two of Mucor racemosus and 5-methylcytosine. According to the “Chinese Clinical Practice Guideline on the Management of Ulcerative Colitis (2023, Xi'an)”, ulcerative colitis is divided into active phase and remission phase according to the severity assessment of ulcerative colitis, where the active phase is further divided into mild, moderate, severe and acute severe phases. The drug for preventing and/or treating ulcerative colitis provided in the present disclosure can target various types of ulcerative colitis, including ulcerative colitis in the remission phase, mild ulcerative colitis, moderate ulcerative colitis, severe ulcerative colitis or acute severe ulcerative colitis. In addition, the dosage form of the drug for preventing and/or treating ulcerative colitis includes an oral formulation, e.g., a lyophilized powder, a tablet, a capsule, a pill, an oral solution, etc.

To make the objective, technical solutions and advantages of the present disclosure clearer, the use of Mucor racemosus and the metabolite thereof provided in the present disclosure in the preparation of a drug for preventing and/or treating ulcerative colitis is described in detail below with reference to examples, which, however, shall not be construed as limiting the scope of protection of the present disclosure.

In the examples of the present disclosure, all data are analyzed statistically using the software GraphPad Prism 8.0, the metrology data are expressed as mean±standard deviation, and inter-group comparison is performed using one-way analysis of variance (One-way ANOVA) or two-way analysis of variance (Two-way ANOVA).

Methods in the following examples are all conventional methods, unless otherwise specified.

The materials, reagents, etc., used in the following examples are all commercially available, unless otherwise specified.

### Example 1 Preparation of Mucor racemosus Fungal Suspension

Mucor racemosus (Mucor racemosus f. racemosus, with the deposit number of CGMCC 3.4941, purchased from the China General Microbiological Culture Collection Center) was inoculated under sterile conditions onto PDA medium covered with cellophane. Then, the culture was statically cultured under aerobic conditions at 30° C. for 48-96 h until the medium was fully covered with Mucor racemosus, and reserved for later use. The PDA medium is potato dextrose agar medium, with components as follows: 6 g/L potato infusion powder, 20 g/L glucose and 20 g/L agar.

Fresh feces from healthy 6-8-week-old C57BL/6J mice were collected. Sterile normal saline was added at a ratio of 0.1 g of feces:1 mL of normal saline, and the mixture was mixed thoroughly. 300 μL of the formulated fecal suspension was spread evenly on PDA+BHI medium. The cellophane fully covered with Mucor racemosus was transferred to the PDA+BHI medium containing the fecal suspension. Then, the culture was statically cultured under strictly anaerobic conditions at 37° C. for 5 days for acclimatization. On the third day of acclimatization, the medium was replaced with fresh PDA+BHI medium spread with fecal suspension. The BHI medium is brain heart infusion broth medium with components as follows: 10 g/L peptone, 12.5 g/L dehydrated calf infusion powder, 5 g/L dehydrated bovine heart infusion powder, 5 g/L sodium chloride, 2 g/L glucose, and 2.5 g/L disodium hydrogen phosphate. The PDA+BHI medium was prepared by mixing PDA medium powder and BHI medium powder at a ratio of 13:4 and sterilizing. Specifically, 39 g of PDA and 52 g of BHI medium were weighed and mixed uniformly, followed by the addition of 1 L of distilled water, and the mixture was sterilized under high temperature and high pressure conditions at 121° C. for 15 min.

On day 5, the acclimated Mucor racemosus was obtained. The acclimated Mucor racemosus was scraped off from the cellophane for deposition, with the deposit number of CGMCC NO: 40320. Sterile normal saline and grinding beads were added to the above Mucor racemosus, followed by grinding. After quantification, a Mucor racemosus fungal suspension with a concentration of 10⁸ CFU/mL was prepared.

### Example 2 Impact of Mucor racemosus on Ulcerative Colitis Mice

### 1. Experimental Materials and Methods

### 1.1 Experimental Materials

Male C57BL/6J mice, 6-8 weeks old, weighing 19-21 grams, were purchased from Beijing Huafukang Biotechnology Co., Ltd. All mice were housed in the SPF Experimental Animal Center, Institute of Radiation Medicine, Chinese Academy of Medical Sciences (Prephase: 22±2° C., humidity: 50-60%, and a 12:12-hour light/dark cycle). The mice were acclimated for one week before the experiment, with ad libitum access to food and water. All animal experiments were performed according to the Guide for the Care and Use of Laboratory Animals, and all operations in the experiments strictly followed the Laboratory Animal Care and Use Manual of Nankai University.

### 1.2 Administration Experimental Regimen

Healthy C57BL/6J mice were randomly divided into three groups by body weight, with six mice in each group.

Model establishment: A mouse model of colitis was established using a 3% mass fraction of dextran sulfate sodium (DSS) solution. Preparation method: Three grams of dextran sulfate sodium powder were weighed, dissolved in 100 mL of sterile water, shaken evenly, and sterilized by filtration through a 0.22 μm filter membrane to obtain a 3% dextran sulfate sodium solution.

Blank group: The mice were given normal drinking water. Administration was started on day 6, with normal saline given by intragastric administration for five consecutive days, at an intragastric dose of 100 μL/mouse/time.

Model group: The mice were given a 3% DSS solution, allowing ad libitum access to the DSS solution for seven days. Administration was started on day 6 of modeling, with normal saline given by intragastric administration for five consecutive days, at an intragastric dose of 100 μL/mouse/time.

Mucor racemosus administration group: The mice were given a 3% DSS solution, allowing ad libitum access to the DSS solution for seven days. Administration was started on day 6 of modeling, with the 10⁸ CFU/mL Mucor racemosus fungal suspension prepared in Example 1 given by intragastric administration for five consecutive days, at an intragastric dose of 100 μL/mouse/time.

After feeding, samples were collected separately. On the basis of different samples and subsequent experimental requirements, the samples were processed and stored for subsequent detection and analysis.

2. Impact of Mucor racemosus on Body Weight of Ulcerative Colitis Mice

Mouse body weight is one of the indicators for judging the severity of ulcerative colitis, which is related to the health status of mice. Specifically, ulcerative colitis causes a significant decrease in mouse body weight. Therefore, the initial body weight of each mouse was recorded before the establishment of mouse model, i.e., on day 0, and the body weight of each mouse was recorded daily after the start of modeling.

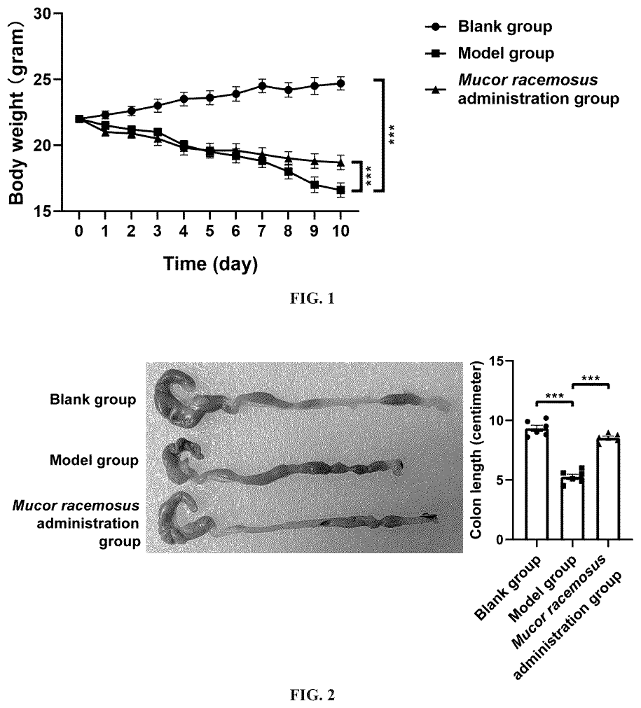

The results are as shown in FIG. 1. Compared to the normal mice in the blank group, the mice in the model group exhibited a significant decrease in body weight 10 days after the start of modeling; compared to the mice in the model group, the mice in the Mucor racemosus administration group exhibited a significant increase in body weight 10 days after the start of modeling, showing a tendency to recover to the body weight of the mice in the blank group. Conclusion: Mucor racemosus can significantly alleviate body weight loss in colitis mice, thus having a certain therapeutic effect on ulcerative colitis.

3. Impact of Mucor racemosus on Colon Length in Ulcerative Colitis Mice

Mouse colon length is one of the indicators for judging the severity of ulcerative colitis. Specifically, ulcerative colitis causes a significant reduction in mouse colon length. Therefore, after being euthanized on day 10, the mice were dissected, and the colon tissues of the mice were collected for length measurement and comparison. The colon length was measured, photographed, and recorded.

The results are as shown in FIG. 2. Compared to the normal mice in the blank group, the mice in the model group exhibited a significant decrease in colon length; compared to the mice in the model group, the mice in the Mucor racemosus administration group exhibited a significant increase in colon length, which was close to that of the mice in the blank group. Conclusion: Mucor racemosus can significantly increase the colon length of colitis mice, thus having an alleviating effect on colon shortening caused by ulcerative colitis.

4. Impact of Mucor racemosus on the Spleen of Ulcerative Colitis Mice

Since ulcerative colitis causes splenomegaly in mice, spleen index (calculated as spleen weight/mouse body weight) is one of the indicators for judging the severity of ulcerative colitis. Specifically, ulcerative colitis causes an increase in the spleen index of mice. Therefore, the mice were weighed on day 10. After being euthanized, the mice were dissected, and the spleen tissues of the mice were weighed to calculate the spleen index.

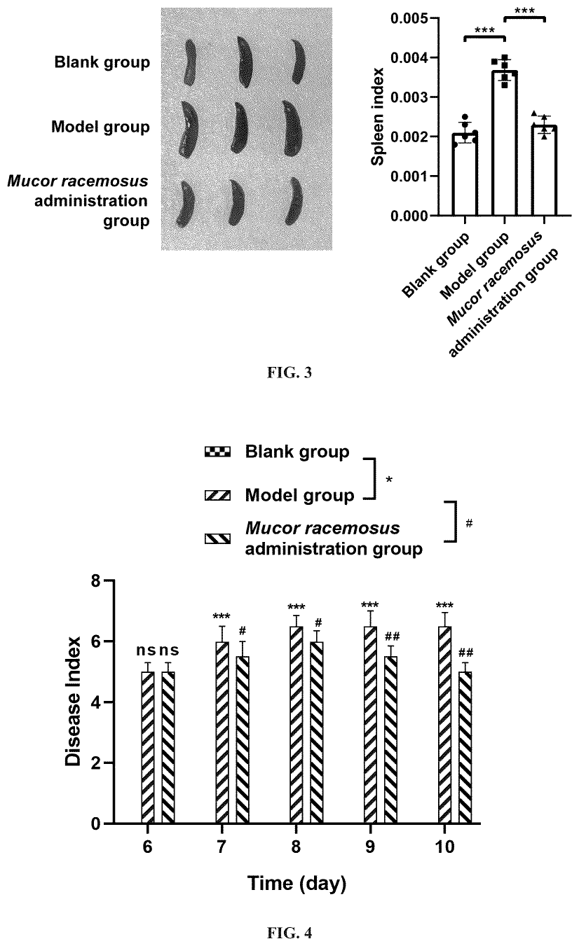

The results are as shown in FIG. 3. Compared to the normal mice in the blank group, the mice in the model group exhibited a significant increase in spleen index; compared to the mice in the model group, the mice in the Mucor racemosus administration group exhibited a significant decrease in spleen index, and the spleen index of the mice in the Mucor racemosus administration group was close to that of the mice in the blank group. Conclusion: Mucor racemosus can significantly reduce the spleen index in colitis mice, thus having a regulatory effect on the immune system.

5. Impact of Mucor racemosus on Disease Index in Ulcerative Colitis Mice

The colitis disease index provides a visual indication for the degree of disease progression of colitis disease in mice. The disease index is a comprehensive score based on three indicators: body weight loss, stool consistency, and bloody stool. From the start of Mucor racemosus administration, i.e., on day 6, the above three indicators were monitored daily and scored according to Table 1. A higher score indicates a more severe disease condition.

TABLE 1
Scoring parameters for disease index

| Score | Body weight loss | Stool consistency | Bloody stool |
|---|---|---|---|
| 0 | None | Normal | Negative |
| 1 | 1-5% | Soft but still formed | Negative |
| 2 | 6-10% | Soft | Positive |
| 3 | 11-18% | Very soft | Visible blood in stool |
| 4 | >18% | Watery diarrhea | Rectal bleeding |

The results are as shown in FIG. 4. The disease index of the mice in the blank group, i.e., the wild-type mice, was always 0; compared to the mice in the blank group, the mice in the model group exhibited a significant increase in disease index with the extension of modeling time; compared to the mice in the model group, the mice in the Mucor racemosus administration group exhibited a significant decrease in disease index with the extension of modeling time. Conclusion: Mucor racemosus can significantly reduce the disease index of colitis mice, thus alleviating the symptoms and severity of colitis disease.

6. Impact of Mucor racemosus on ROS Content in Colon of Ulcerative Colitis Mice

Colitis disease results in the production of large amounts of ROS in colon tissue cells. Excessive ROS causes DNA mutations and mitochondrial dysfunction, thereby damaging biomolecules and cells and inducing cell death. In addition, ROS is a primary mediator of oxidative stress in the body. When the production of ROS exceeds the antioxidant capacity of cells, an oxidative stress state is induced, which damages cellular macromolecules (such as lipids, proteins and DNA). Therefore, ROS content in colon tissue was determined to detect the impact of Mucor racemosus on the ROS content in colon tissue. After being euthanized on day 10, the mice were dissected, and the colon tissues of the mice were collected. An appropriate amount of normal saline was added to the tissues. Specifically, 1 mL of normal saline was added per 0.1 g of tissue. The tissues were homogenized and centrifuged at 3000 g/min for 20 minutes, and the supernatant was collected. The ROS content in the supernatant was detected and determined using the ROS assay kit (Enzyme-linked Biotechnology, China).

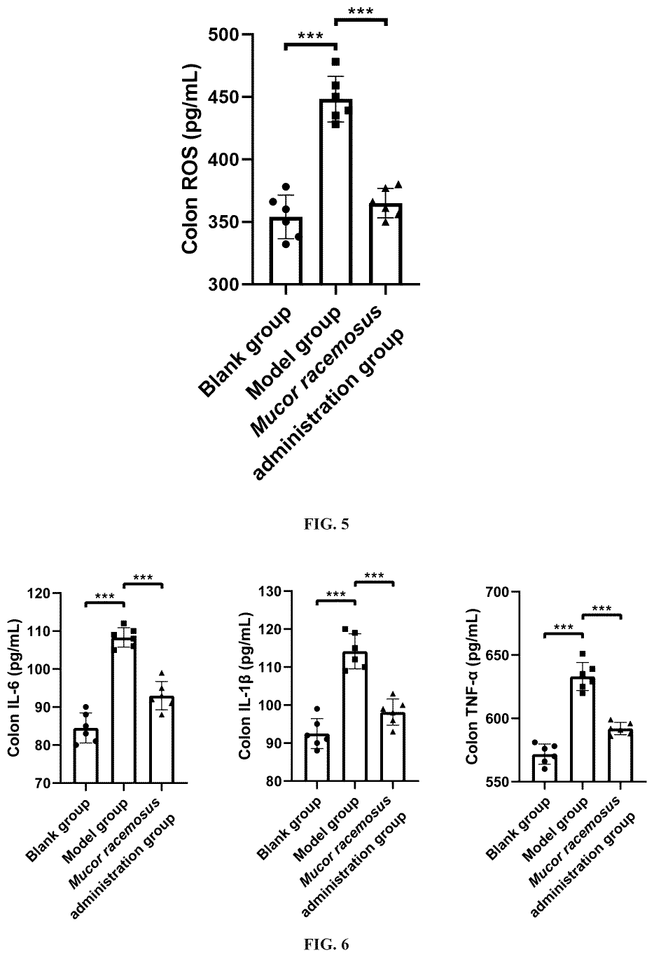

The results are as shown in FIG. 5. Compared to the normal mice in the blank group, the mice in the model group exhibited a significant increase in ROS content in the colon; compared to the mice in the model group, the mice in the Mucor racemosus administration group exhibited a significant decrease in ROS content in the colon, which was close to that of the mice in the blank group. Conclusion: Mucor racemosus can significantly reduce the ROS content in colon tissue of colitis mice, reflecting that Mucor racemosus has a certain antioxidant capacity.

7. Impact of Mucor racemosus on Contents of IL-6, IL-1β and TNF-α in Colon of Ulcerative Colitis Mice

IL-6, IL-1β and TNF-α are indicators for judging colitis disease, reflecting the degree of progression of inflammation in mouse colon tissue. Therefore, the contents of IL-6, IL-1β and TNF-α in colon tissue were determined to detect the impact of Mucor racemosus on inflammation in colon tissue. The contents of three factors in colon tissue were determined using the IL-6 assay kit, IL-1β assay kit and TNF-α assay kit (Enzyme-linked Biotechnology, China), respectively.

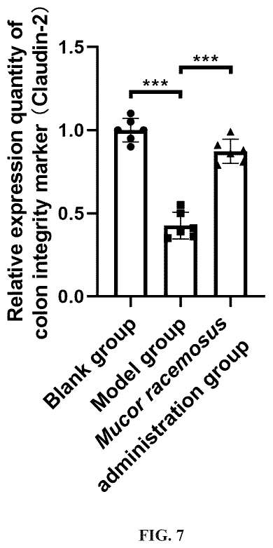

The results are as shown in FIG. 6. Compared to the mice in the blank group, the mice in the model group exhibited a significant increase in the contents of IL-6, IL-1β and TNF-α in the colon; compared to the mice in the model group, the mice in the Mucor racemosus administration group exhibited a significant decrease in the contents of IL-6, IL-1β and TNF-α in the colon, which was closed to those of the mice in the blank group. Conclusion: Mucor racemosus can significantly reduce the contents of IL-6, IL-1β and TNF-α in colon tissues of colitis mice, thus exerting an anti-inflammatory effect. This confirms that Mucor racemosus treats ulcerative colitis from the perspective of anti-inflammation.

8. Impact of Mucor racemosus on Expression Quantity of Claudin-2, a Marker of Intestinal Wall Integrity of Colon, in Colitis Mice

Colitis disease causes serious damage to mouse colon tissue. Specifically, the intestinal wall integrity of colon is disrupted. Therefore, the colon wall integrity can be judged by detecting the expression quantity of Claudin-2 (tight junction protein-2), a marker of intestinal wall integrity of colon.

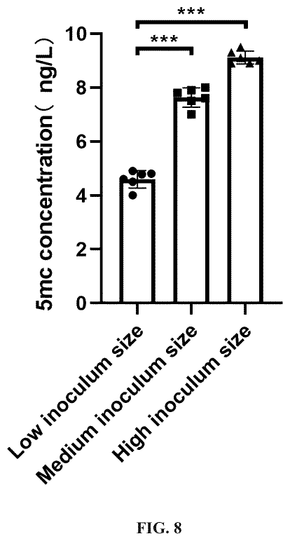

The results are as shown in FIG. 7. Compared to the normal mice in the blank group, the mice in the model group exhibited a significant decrease in the content of Claudin-2 in the colon; compared to the mice in the model group, the mice in the Mucor racemosus administration group exhibited a significant increase in the content of Claudin-2 in the colon, which was close to that of the mice in the blank group. Conclusion: Mucor racemosus can significantly enhance the intestinal wall integrity of colon tissue of colitis mice, thus effectively delaying the progression of ulcerative colitis.

### Example 3 Detection of Metabolite 5-Methylcytosine Produced by Mucor racemosus In Vivo and In Vitro

1. Propagation Culture of Mucor racemosus with Different Inoculum Sizes

PDB was mixed with BHI medium powder at a ratio of 6:13 and sterilized to prepare PDB+BHI liquid medium. Specifically, 24 g of PDB and 52 g of BHI medium were weighed and mixed uniformly, followed by the addition of 1 L of distilled water, and the mixture was sterilized under high temperature and high pressure conditions at 121° C. for 15 min. The Mucor racemosus fungal suspension prepared in Example 1 was inoculated into the PDB+BHI liquid medium at different inoculum sizes, specifically at inoculation units of 5×10⁶/100 mL, 10⁷/100 mL and 2×10⁷/100 mL, respectively. The cultures were cultured under strictly anaerobic conditions at 37° C. for 3 days to obtain three groups of Mucor racemosus fungal suspensions with low, medium, and high inoculum sizes, respectively. The PDB medium is potato dextrose medium, with components as follows: 6 g/L potato infusion powder and 20 g/L glucose.

### 2. Detection

The contents of 5-methylcytosine produced by Mucor racemosus in the host and in in-vitro culture were determined using the 5-methylcytosine assay kit (Enzyme-linked Biotechnology, China).

The above three groups of Mucor racemosus fungal suspensions with different inoculum sizes were collected and centrifuged at 3000 g/min for 20 minutes, and the supernatants were collected. In addition, the feces from the mice in the blank group, model group and Mucor racemosus administration group of Example 1 were collected on day 10. An appropriate amount of normal saline was added to the mouse feces. Specifically, 1 mL of normal saline was added per 0.1 g of feces. The feces were homogenized and centrifuged at 3000 g/min for 20 minutes, and the supernatants were collected.

The above six groups of supernatants were diluted with a sample diluent at a ratio of 1:1. Then, 50 μL of each of the diluted supernatants was added into a reaction well, 50 μL of the diluted standard was added into a reaction well, and 50 μL of each of the above supernatant samples was added into a reaction well. 50 μL of biotin-labeled antibody was immediately added. The microplate was covered, shaken gently for uniform mixing, and then incubated at 37° C. for 1 hour. The liquid in the wells was discarded. Each well was filled with a washing solution, and then the microplate was shaken for 30 seconds. The washing solution was discarded, and then the microplate was patted dry with absorbent paper. This operation was repeated three times. If washing was performed with a plate washer, the number of washing cycles was increased by one. 80 μL of streptavidin-HRP was added to each well. The microplate was shaken gently for uniform mixing, and then incubated at 37° C. for 30 minutes. The liquid in the wells was discarded. Each well was filled with a washing solution, and then the microplate was shaken for 30 seconds. The washing solution was discarded, and then the microplate was patted dry with absorbent paper. This operation was repeated three times. If washing was performed with a plate washer, the number of washing cycles was increased by one. 50 μL each of substrates A and B to each well. The microplate was shaken gently for uniform mixing, and then incubated at 37° C. for 10 minutes. Light exposure was avoided. The microplate was taken out, and 50 μL of stop solution was quickly added. The results should be measured immediately after the addition of the stop solution. The OD value of each well was determined at a wavelength of 450 nm. Taking the absorbance OD value as the ordinate (Y) and the corresponding concentration of the standard solution of the substance to be detected as the abscissa (X), a corresponding curve was plotted. The content of the substance to be detected in the sample could be converted to the corresponding concentration from the standard curve according to its OD value.

The results are as shown in FIG. 8 and FIG. 9. When Mucor racemosus was cultured in liquid medium in vitro, the amount of 5-methylcytosine produced increased significantly with the increase in the inoculum size of Mucor racemosus. This indicates that 5-methylcytosine is a metabolite of Mucor racemosus. In addition, it was found in in-vivo animal experiments that compared to the mice in the blank group, the mice in the model group exhibited a significant decrease in the content of 5-methylcytosine in the feces. Compared to the mice in the model group, the mice in the Mucor racemosus administration group exhibited a significant increase in the content of 5-methylcytosine in the feces, which was close to that of the mice in the blank group. Therefore, Mucor racemosus can metabolically produce 5-methylcytosine both in the host and in in-vitro culture. This also confirms that 5-methylcytosine is a metabolite of Mucor racemosus.

### Example 4 Impact of 5mc, a Metabolite of Mucor racemosus, on Ulcerative Colitis Mice

### 1. Experimental Materials and Methods

### 1.1 Experimental Materials

The experimental materials were the same to those described in the section “1.1 Experimental materials” of Example 2.

### 1.2 Experimental Methods

Healthy C57BL/6J mice were randomly divided into three groups by body weight, namely, the blank group, the model group, and the 5mc intragastric administration group, with six mice in each group.

The model establishment and administration methods were the same as those described in the section “1.2 Administration experimental regimen” of Example 2, except that the 5mc intragastric administration group was given 2 mg/mL 5mc by intragastric administration starting from day 6 of modeling, for five consecutive days, at an intragastric dose of 100 μL/mouse/time.

After 10 days of feeding, samples were collected separately. On the basis of different samples and subsequent experimental requirements, the samples were processed and stored for subsequent detection and analysis.

### 2. Detection

The detection indicators and operating methods were the same as those described in sections “2-8” of Example 2, which specifically included the detection items of mouse body weight, colon length, disease index, contents of ROS and inflammatory factors (IL-6, IL1-β and TNF-α), and expression quantity of Claudin-2.

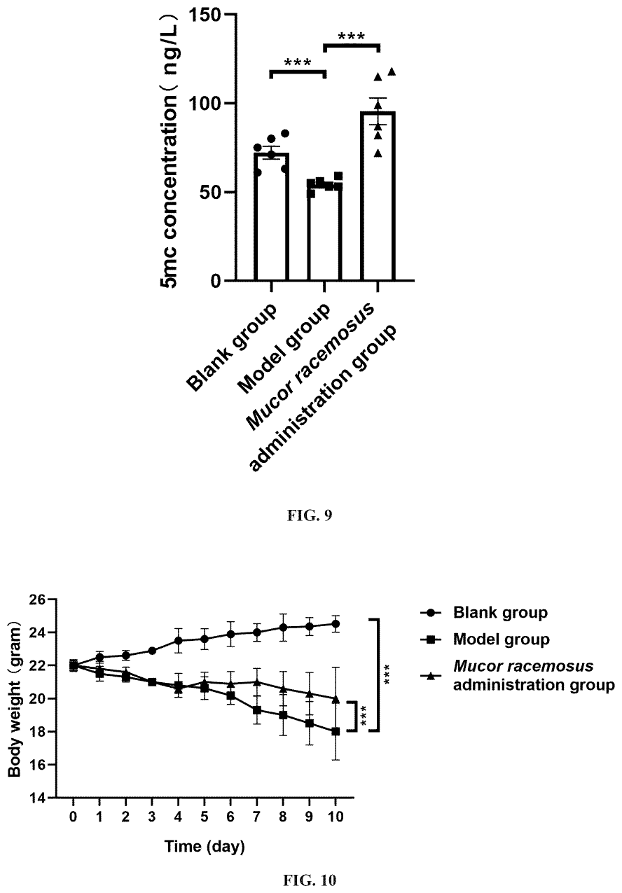

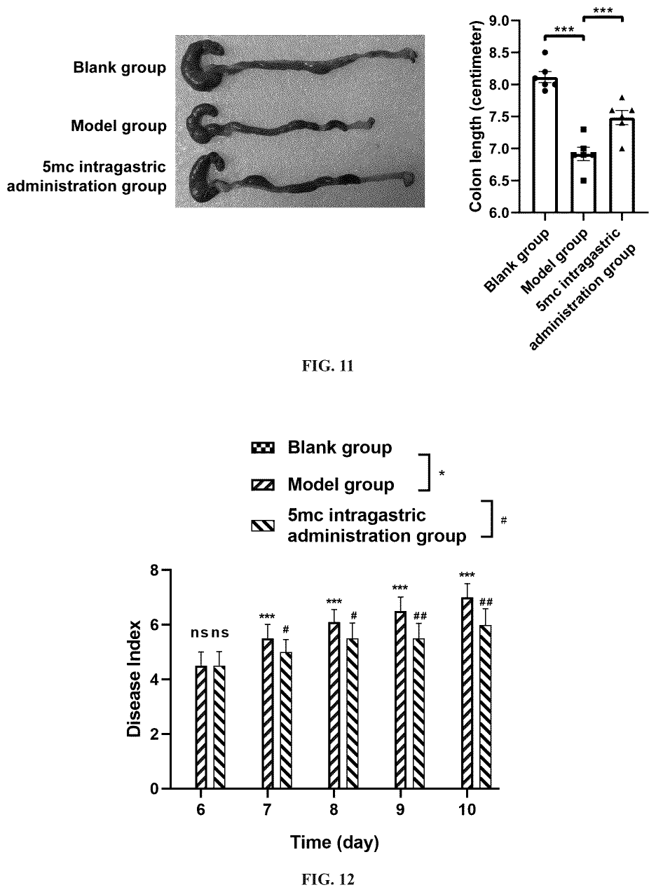

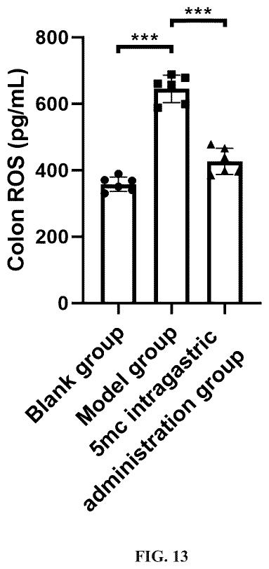

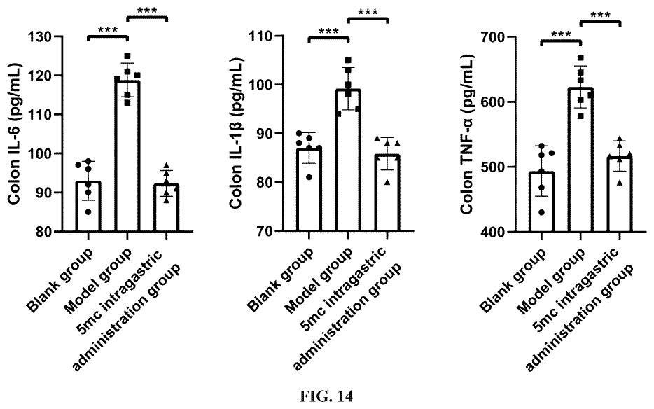

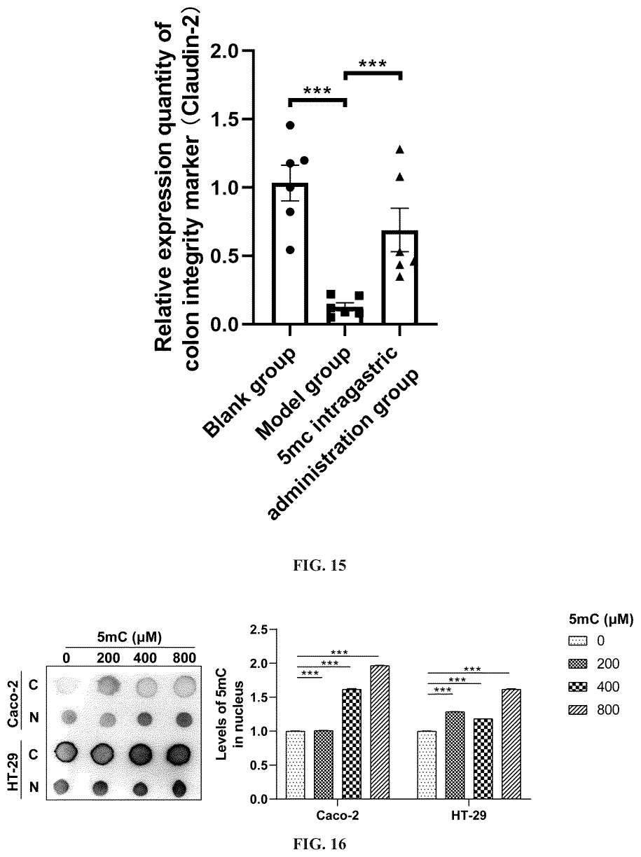

The results are as shown in FIGS. 10-15. Compared with the normal mice in the blank group, the mice in the model group exhibited a significant decrease in body weight and a significant shortening in colon length. Moreover, with the extension of modeling time, the disease development index of the mice increased significantly, the ROS content and the contents of inflammation-related factors IL-6, IL1-β and TNF-α in colon tissue increased significantly, and the expression quantity of Claudin-2, a marker gene of intestinal wall integrity of colon, decreased significantly. However, compared to the mice in the model group, the mice in the 5-methylcytosine intragastric administration group exhibited a significant increase in body weight and a significant increase in colon length. Moreover, with the extension of modeling time, the disease development index decreased significantly, the ROS content and the contents of inflammation-related factors IL-6, IL1-β and TNF-α in colon tissue decreased significantly, and the expression quantity of Claudin-2, a marker gene of intestinal wall integrity of colon, increased significantly. Conclusion: The metabolite 5-methylcytosine of Mucor racemosus can effectively treat ulcerative colitis disease.

### Example 5 Accessibility of Exogenous 5mc to the Host Colon Cell Nucleus

### 1. Experimental Materials and Methods

### 1.1 Experimental Materials

The human colon cell lines (Caco-2 and HT-29) were both from the American Type Culture Collection (ATCC). The two cell lines were cultured in MEM (containing 10% fetal bovine serum (Gibco) and 10 U/mL penicillin-streptomycin (Gibco)) and DMEM medium, respectively. The cells were cultured in an incubator at 37° C. with 5% CO₂.

### 1.2 Experimental Methods

Exogenous 5mc at different concentrations (0 μM, 200 μM, 400 μM and 800 μM) was co-incubated with the human colon cell lines Caco-2 and HT-29 for 48 h. The cytoplasm and nucleus were extracted separately using the NE-PER Nuclear and Cytoplasmic Extraction Reagents kit from Thermo Fisher Scientific. The contents of 5mc in the cytoplasm and nucleus were detected by Dot blot, with the procedures as follows:
1.2.1 A nitrocellulose membrane was prepared. a grid was drawn with a pencil to mark the areas to be blotted.
1.2.2 Using a narrow-mouth pipette tip, 2 μL of sample was spotted onto the nitrocellulose membrane at the center of the grid. The sample was added dropwise slowly.
1.2.3 The membrane was allowed to dry.
1.2.4 The membrane was immersed in a solution of 5% BSA in TBS-T in a 10 cm Petri dish (at room temperature for 30 minutes to 1 hour) to block non-specific sites.
1.2.5 The 5mc primary antibody (Abcam, Cat No. ab214727) was dissolved in BSA/TBS-T buffer and incubated at room temperature for 30 minutes.
1.2.6 Washing was performed three times with TBS-T buffer (3 times, 5 minutes each).
1.2.7 The rabbit secondary antibody was dissolved in BSA/TBS-T buffer and incubated at room temperature for 30 minutes.
1.2.8 Washing was performed three times with TBS-T buffer (once for 15 minutes and twice for 5 minutes each time), and then once with TBS buffer (5 minutes).
1.2.9 The secondary antibody was incubated with ECL reagent for 1 minute. The membrane was then covered with plastic wrap (to remove excess solution on the surface) and exposed to an X-ray film in a darkroom.
1.2.10 The relative content of 5mc was calculated.

### 2. Detection

The results are shown in FIG. 16. The relative contents of 5mC in the cytoplasm and nucleus were quantified using imageJ software. The results showed that when two colon cell lines, Caco-2 and HT-29, were treated with different concentrations of exogenous 5mc, the concentrations of 5mc in both the cytoplasm and nucleus increased significantly as the concentration of exogenous 5mc increased. Therefore, exogenous 5mc can enter the host colon cell nucleus.

### Example 6 Ability of Exogenous 5mc to Regulate Gene Expression in Host Colon Cells

### 1. Experimental Materials and Methods

Promoter sequences with different GC ratios (BAZIA, 45% GC; ZHX2, 56% GC; MYC, 66% GC; NFKBIE, 74% GC) were cloned into the multiple cloning site of the pGL3-Basic luciferase reporter plasmid. Caco-2 and HT-29 cells were seeded in a 6-well plate and co-transfected with a DNA mixture containing 5 μg firefly luciferase reporter plasmid (pGL3-Basic) and 0.5 μg of Renilla luciferase plasmid (pRL-TK) per well. Moreover, 0 and 800 μM 5mc were added to the cells. After 18 hours, luciferase activity was determined using the luciferase assay kit (Promega, USA, Cat. No. E1910) according to the manufacturer's instructions.

### 2. Detection

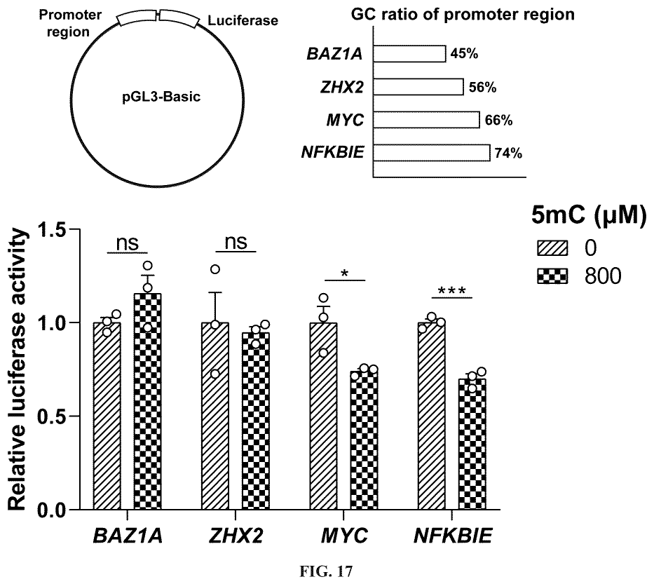

The results are shown in FIG. 17. It is seen that 5mc had a relatively small impact on the expression of genes with a low GC ratio in the promoter region, such as BAZIA and ZHX2; 5mc could significantly inhibit the expression of genes with a high GC ratio in the promoter region, such as MYC and NFKBIE, and MYC and NFKBIE were involved in the occurrence of a variety of diseases such as inflammation and tumors. Therefore, exogenous 5mc can regulate gene expression in host colon cells, and this regulation is related to the GC ratio in the promoter region of the gene. Specifically, the higher the GC ratio, the more likely the expression of genes is inhibited. Conclusion: The metabolite 5-methylcytosine of Mucor racemosus can effectively treat ulcerative colitis disease. This is because 5-methylcytosine can enter the host cell nucleus and inhibit the expression of inflammation-related genes.

The coding sequences of the major components of the luciferase expression plasmids are listed below:

pGL3-Basic sequence (5′-3′):
(SEQ ID NO: 1)
GGTACCGAGCTCTTACGCGTGCTAGCCCGGGCTCGAGATCTGCGA
TCTAAGTAAGCTTGGCATTCCGGTACTGTTGGTAAAGCCACCATG
GAAGACGCCAAAAACATAAAGAAAGGCCCGGCGCCATTCTATCCG
CTGGAAGATGGAACCGCTGGAGAGCAACTGCATAAGGCTATGAAG
AGATACGCCCTGGTTCCTGGAACAATTGCTTTTACAGATGCACAT
ATCGAGGTGGACATCACTTACGCTGAGTACTTCGAAATGTCCGTT
CGGTTGGCAGAAGCTATGAAACGATATGGGCTGAATACAAATCAC
AGAATCGTCGTATGCAGTGAAAACTCTCTTCAATTCTTTATGCCG
GTGTTGGGCGCGTTATTTATCGGAGTTGCAGTTGCGCCCGCGAAC
GACATTTATAATGAACGTGAATTGCTCAACAGTATGGGCATTTCG
CAGCCTACCGTGGTGTTCGTTTCCAAAAAGGGGTTGCAAAAAATT
TTGAACGTGCAAAAAAAGCTCCCAATCATCCAAAAAATTATTATC
ATGGATTCTAAAACGGATTACCAGGGATTTCAGTCGATGTACACG
TTCGTCACATCTCATCTACCTCCCGGTTTTAATGAATACGATTTT
GTGCCAGAGTCCTTCGATAGGGACAAGACAATTGCACTGATCATG
AACTCCTCTGGATCTACTGGTCTGCCTAAAGGTGTCGCTCTGCCT
CATAGAACTGCCTGCGTGAGATTCTCGCATGCCAGAGATCCTATT
TTTGGCAATCAAATCATTCCGGATACTGCGATTTTAAGTGTTGTT
CCATTCCATCACGGTTTTGGAATGTTTACTACACTCGGATATTTG
ATATGTGGATTTCGAGTCGTCTTAATGTATAGATTTGAAGAAGAG
CTGTTTCTGAGGAGCCTTCAGGATTACAAGATTCAAAGTGCGCTG
CTGGTGCCAACCCTATTCTCCTTCTTCGCCAAAAGCACTCTGATT
GACAAATACGATTTATCTAATTTACACGAAATTGCTTCTGGTGGC
GCTCCCCTCTCTAAGGAAGTCGGGGAAGCGGTTGCCAAGAGGTTC
CATCTGCCAGGTATCAGGCAAGGATATGGGCTCACTGAGACTACA
TCAGCTATTCTGATTACACCCGAGGGGGATGATAAACCGGGCGCG
GTCGGTAAAGTTGTTCCATTTTTTGAAGCGAAGGTTGTGGATCTG
GATACCGGGAAAACGCTGGGCGTTAATCAAAGAGGCGAACTGTGT
GTGAGAGGTCCTATGATTATGTCCGGTTATGTAAACAATCCGGAA
GCGACCAACGCCTTGATTGACAAGGATGGATGGCTACATTCTGGA
GACATAGCTTACTGGGACGAAGACGAACACTTCTTCATCGTTGAC
CGCCTGAAGTCTCTGATTAAGTACAAAGGCTATCAGGTGGCTCCC
GCTGAATTGGAATCCATCTTGCTCCAACACCCCAACATCTTCGAC
GCAGGTGTCGCAGGTCTTCCCGACGATGACGCCGGTGAACTTCCC
GCCGCCGTTGTTGTTTTGGAGCACGGAAAGACGATGACGGAAAAA
GAGATCGTGGATTACGTCGCCAGTCAAGTAACAACCGCGAAAAAG
TTGCGCGGAGGAGTTGTGTTTGTGGACGAAGTACCGAAAGGTCTT
ACCGGAAAACTCGACGCAAGAAAAATCAGAGAGATCCTCATAAAG
GCCAAGAAGGGCGGAAAGATCGCCGTGTAATTCTAGAGTCGGGGC
GGCCGGCCGCTTCGAGCAGACATGATAAGATACATTGATGAGTTT
GGACAAACCACAACTAGAATGCAGTGAAAAAAATGCTTTATTTGT
GAAATTTGTGATGCTATTGCTTTATTTGTAACCATTATAAGCTGC
AATAAACAAGTTAACAACAACAATTGCATTCATTTTATGTTTCAG
GTTCAGGGGGAGGTGTGGGAGGTTTTTTAAAGCAAGTAAAACCTC
TACAAATGTGGTAAAATCGATAAGGATCCGTCGACCGATGCCCTT
GAGAGCCTTCAACCCAGTCAGCTCCTTCCGGTGGGCGCGGGGCAT
GACTATCGTCGCCGCACTTATGACTGTCTTCTTTATCATGCAACT
CGTAGGACAGGTGCCGGCAGCGCTCTTCCGCTTCCTCGCTCACTG
ACTCGCTGCGCTCGGTCGTTCGGCTGCGGCGAGCGGTATCAGCTC
ACTCAAAGGCGGTAATACGGTTATCCACAGAATCAGGGGATAACG
CAGGAAAGAACATGTGAGCAAAAGGCCAGCAAAAGGCCAGGAACC
GTAAAAAGGCCGCGTTGCTGGCGTTTTTCCATAGGCTCCGCCCCC
CTGACGAGCATCACAAAAATCGACGCTCAAGTCAGAGGTGGCGAA
ACCCGACAGGACTATAAAGATACCAGGCGTTTCCCCCTGGAAGCT
CCCTCGTGCGCTCTCCTGTTCCGACCCTGCCGCTTACCGGATACC
TGTCCGCCTTTCTCCCTTCGGGAAGCGTGGCGCTTTCTCATAGCT
CACGCTGTAGGTATCTCAGTTCGGTGTAGGTCGTTCGCTCCAAGC
TGGGCTGTGTGCACGAACCCCCCGTTCAGCCCGACCGCTGCGCCT
TATCCGGTAACTATCGTCTTGAGTCCAACCCGGTAAGACACGACT
TATCGCCACTGGCAGCAGCCACTGGTAACAGGATTAGCAGAGCGA
GGTATGTAGGCGGTGCTACAGAGTTCTTGAAGTGGTGGCCTAACT
ACGGCTACACTAGAAGAACAGTATTTGGTATCTGCGCTCTGCTGA
AGCCAGTTACCTTCGGAAAAAGAGTTGGTAGCTCTTGATCCGGCA
AACAAACCACCGCTGGTAGCGGTGGTTTTTTTGTTTGCAAGCAGC
AGATTACGCGCAGAAAAAAAGGATCTCAAGAAGATCCTTTGATCT
TTTCTACGGGGTCTGACGCTCAGTGGAACGAAAACTCACGTTAAG
GGATTTTGGTCATGAGATTATCAAAAAGGATCTTCACCTAGATCC
TTTTAAATTAAAAATGAAGTTTTAAATCAATCTAAAGTATATATG
AGTAAACTTGGTCTGACAGTTACCAATGCTTAATCAGTGAGGCAC
CTATCTCAGCGATCTGTCTATTTCGTTCATCCATAGTTGCCTGAC
TCCCCGTCGTGTAGATAACTACGATACGGGAGGGCTTACCATCTG
GCCCCAGTGCTGCAATGATACCGCGAGACCCACGCTCACCGGCTC
CAGATTTATCAGCAATAAACCAGCCAGCCGGAAGGGCCGAGCGCA
GAAGTGGTCCTGCAACTTTATCCGCCTCCATCCAGTCTATTAATT
GTTGCCGGGAAGCTAGAGTAAGTAGTTCGCCAGTTAATAGTTTGC
GCAACGTTGTTGCCATTGCTACAGGCATCGTGGTGTCACGCTCGT
CGTTTGGTATGGCTTCATTCAGCTCCGGTTCCCAACGATCAAGGC
GAGTTACATGATCCCCCATGTTGTGCAAAAAAGCGGTTAGCTCCT
TCGGTCCTCCGATCGTTGTCAGAAGTAAGTTGGCCGCAGTGTTAT
CACTCATGGTTATGGCAGCACTGCATAATTCTCTTACTGTCATGC
CATCCGTAAGATGCTTTTCTGTGACTGGTGAGTACTCAACCAAGT
CATTCTGAGAATAGTGTATGCGGCGACCGAGTTGCTCTTGCCCGG
CGTCAATACGGGATAATACCGCGCCACATAGCAGAACTTTAAAAG
TGCTCATCATTGGAAAACGTTCTTCGGGGCGAAAACTCTCAAGGA
TCTTACCGCTGTTGAGATCCAGTTCGATGTAACCCACTCGTGCAC
CCAACTGATCTTCAGCATCTTTTACTTTCACCAGCGTTTCTGGGT
GAGCAAAAACAGGAAGGCAAAATGCCGCAAAAAAGGGAATAAGGG
CGACACGGAAATGTTGAATACTCATACTCTTCCTTTTTCAATATT
ATTGAAGCATTTATCAGGGTTATTGTCTCATGAGCGGATACATAT
TTGAATGTATTTAGAAAAATAAACAAATAGGGGTTCCGCGCACAT
TTCCCCGAAAAGTGCCACCTGACGCGCCCTGTAGCGGCGCATTAA
GCGCGGCGGGTGTGGTGGTTACGCGCAGCGTGACCGCTACACTTG
CCAGCGCCCTAGCGCCCGCTCCTTTCGCTTTCTTCCCTTCCTTTC
TCGCCACGTTCGCCGGCTTTCCCCGTCAAGCTCTAAATCGGGGGC
TCCCTTTAGGGTTCCGATTTAGTGCTTTACGGCACCTCGACCCCA
AAAAACTTGATTAGGGTGATGGTTCACGTAGTGGGCCATCGCCCT
GATAGACGGTTTTTCGCCCTTTGACGTTGGAGTCCACGTTCTTTA
ATAGTGGACTCTTGTTCCAAACTGGAACAACACTCAACCCTATCT
CGGTCTATTCTTTTGATTTATAAGGGATTTTGCCGATTTCGGCCT
ATTGGTTAAAAAATGAGCTGATTTAACAAAAATTTAACGCGAATT
TTAACAAAATATTAACGCTTACAATTTGCCATTCGCCATTCAGGC
TGCGCAACTGTTGGGAAGGGCGATCGGTGCGGGCCTCTTCGCTAT
TACGCCAGCCCAAGCTACCATGATAAGTAAGTAATATTAAGGTAC
GGGAGGTACTTGGAGCGGCCGCAATAAAATATCTTTATTTTCATT
ACATCTGTGTGTTGGTTTTTTGTGTGAATCGATAGTACTAACATA
CGCTCTCCATCAAAACAAAACGAAACAAAACAAACTAGCAAAATA
GGCTGTCCCCAGTGCAAGTGCAGGTGCCAGAACATTTCTCTATCG
ATA.
BAZ1A promoter region sequence (5′-3′):
(SEQ ID NO: 2)
CTAGATCTTTTGGCTGATAATCTCAAACATGGAGGATGCTTCTGA
ATCTTCACGAGGGGTTGCTCCATTAATTAATAATGTAGTTCTCCC
AGGCTCTCCGCTGTCTCTTCCTGTATCAGTGACAGGCTGTAAAAG
TCATCGAGTAGCCAATAAAAAGGTAGAAGCGAGGAGTGAAAAGCT
CCTCCCAACAGCTCTTCCTC.
ZHX2 promoter region sequence (5′-3′):
(SEQ ID NO: 3)
ATTTACTGCCCCCCTACCTTGGCCAGTAACCAGCGCCTTTAGGGC
TAGCCTCCCCCCCAGCTCCTGCCTGAAAAATGACATTTCGCCGGT
GTCTCCGGAGGGGGCTGAATTTCACTTTGTAACTTTCTGCGGAAC
CCGAGCCCGGGTGGCAGCTCGGGTGGTGGTATCGTATGCAAATAC
GCATGCTGACGTTACAGATC.
MYC promoter region sequence (5′-3′):
(SEQ ID NO: 4)
CACCGGCCCTTTATAATGCGAGGGTCTGGACGGCTGAGGACCCCC
GAGCTGTGCTGCTCGCGGCCGCCACCGCCGGGCCCCGGCCGTCCC
TGGCTCCCCTCCTGCCTCGAGAAGGGCAGGGCTTCTCAGAGGCTT
GGCGGGAAAAAGAACGGAGGGAGGGATCGCGCTGAGTATAAAAGC
CGGTTTTCGGGGCTTTATCT.
NFKBIE promoter region sequence (5′-3′):
(SEQ ID NO: 5)
AGGAGTGAGTCAAGGCCCGGGAACCACAGACTCCAAGCCTACGCA
GAGCCCGGGAAGGGGGATTCCGGAGGGGCGGGGCCTCTTTCCGGA
AGCGCCCGCCGGGGGCGGGGAGGGGGCGGGGCCATCCGCGTGAGG
CGACCCTGTTGGTCCGGAGGGGCGGGGCGAGGAGGAGGACCCGCT
TGGGCGGTTCGGCTGCCCAC.

The descriptions above are merely the preferred embodiments of the present disclosure. It should be noted that several improvements and modifications may also be made by those of ordinary skill in the art without departing from the principle of the present disclosure, and these improvements and modifications shall also be considered within the scope of protection of the present disclosure.

What is claimed is:

1. A method for preventing and/or treating ulcerative colitis, comprising administering a drug to a subject in need thereof, wherein the drug is Mucor racemosus or a formulation containing Mucor racemosus, and wherein Mucor racemosus is deposited in the China General Microbiological Culture Collection Center (CGMCC) with the deposit number of CGMCC NO: 40320.

2. A method for preventing and/or treating ulcerative colitis, comprising administering a drug to a subject in need thereof, wherein the drug is a metabolite of Mucor racemosus or a formulation containing the metabolite of Mucor racemosus, and wherein the metabolite of Mucor racemosus includes 5-methylcytosine.

3. The method according to claim 2, wherein the drug is any one or more of the following drugs:
1) a drug for alleviating body weight loss in an ulcerative colitis patient;
2) a drug for increasing colon length of an ulcerative colitis patient;
3) a drug having an immunomodulatory effect;
4) a drug having an anti-inflammatory effect;
5) a drug having an antioxidant capacity; and
6) a drug for enhancing intestinal wall integrity of colon tissue.

4. The method according to claim 3, wherein the drug having an immunomodulatory effect in 3) can reduce spleen index in an ulcerative colitis patient.

5. The method according to claim 3, wherein the drug having an anti-inflammatory effect in 4) can reduce a content of any one or several of IL-6, IL-1β and TNF-α in ulcerative colitis tissue.

6. The method according to claim 3, wherein the drug having an antioxidant capacity in 5) can reduce a content of ROS in ulcerative colitis tissue.

7. The method according to claim 3, wherein The drug for enhancing intestinal wall integrity of colon tissue in 6) can increase an expression quantity of Claudin-2 in ulcerative colitis tissue.

8. A drug for preventing and/or treating ulcerative colitis, wherein the drug includes one or two of Mucor racemosus and 5-methylcytosine.

9. The drug for preventing and/or treating ulcerative colitis according to claim 8, wherein the ulcerative colitis includes ulcerative colitis in remission phase, mild ulcerative colitis, moderate ulcerative colitis, severe ulcerative colitis or acute severe ulcerative colitis.

10. The drug for preventing and/or treating ulcerative colitis according to claim 8, wherein the drug includes an oral formulation.

11. The method according to claim 1, wherein the drug is any one or more of the following drugs:
1) a drug for alleviating body weight loss in an ulcerative colitis patient;
2) a drug for increasing colon length of an ulcerative colitis patient;
3) a drug having an immunomodulatory effect;
4) a drug having an anti-inflammatory effect;
5) a drug having an antioxidant capacity; and
6) a drug for enhancing intestinal wall integrity of colon tissue.
